(19) United States
(12) Patent Application Publication
(10) Pub. No.: US 2017/0055958 A1
(43) Pub. Date: Mar. 2, 2017

(54) Urine Diagnostic Testing Device

(71) Applicant: Jesse Suarez, West Covina, CA (US)

(72) Inventor: Jesse Suarez, West Covina, CA (US)

(21) Appl. No.: 15/247,347

(22) Filed: Aug. 25, 2016

Related U.S. Application Data
(60) Provisional application No. 62/211,273, filed on Aug. 28, 2015.

(51) Int. Cl. A61B 10/00; A61M 39/24; A61B 5/145; A61M 25/00

(52) U.S. Cl. CPC A61B 10/007; A61M 25/0017; A61M 39/24; A61B 5/14507; A61B 2010/0009; A61B 2562/0295

(57) ABSTRACT

A urine toxicology testing device. The urine toxicology testing device includes a collection container having one or more toxicology testing strips for completing a chemical analysis of the collected urine. The testing strips are positioned within a drum that can be placed within the collection container and exposed to collected urine. A catheter has a first end that is in fluid communication with an opposing second end, wherein the first end is configured to be inserted into a urethra. The second end is disposed within the collection container, through a lid affixed to the collection container. The lid includes a valve that is movable between an open and closed position.

### CROSS REFERENCE TO RELATED APPLICATION

This application claims the benefit of U.S. Provisional Application No. 62/211,273 filed on Aug. 28, 2015. The above identified patent application is herein incorporated by reference in its entirety to provide continuity of disclosure.

### BACKGROUND OF THE INVENTION

The present invention relates to a urine testing device. More specifically, the present invention relates to a urine testing a device that utilizes a catheter to collect urine within a collection container having one or more toxicology testing strips for completing a chemical analysis of the collected urine.

A toxicology screen is often performed in emergency medical situations to determine if patient has certain chemicals or metabolites within their system. Typically, the toxicology screen requires the patient to provide a urine sample that is sent to a laboratory where the urine toxicology analysis is performed. To perform the toxicology analysis, a test strip having reagent chemicals impregnated into them is contacted with the urine. The urine is absorbed into the test stripes and a chemical reaction occurs between the reagents and urine, typically producing a visual indication of the chemical reaction, such as a change in color of the test strip. The test strip is compared to a color chart having the results of each test. The evaluation of the test alp with the color chart produces a negative test result if the urine does not contain a sufficient amount of certain chemical or metabolite. A positive test result is produced if the urine does contain a sufficient amount of certain chemical or metabolite.

However, there are several deficiencies with the above stated toxicology screen. First, obtaining urine from certain patients, for example unconscious patients, requires catheterization of the individual and collection of urine to a collection bag. This current testing process requires transferring the urine from a collection bag to a toxicology collection container so that the test strips may be tested. In some circumstances, delays in receiving test results delays treatment which may have significant impact on patient treatment and outcome. This is especially true for a patient with a suspected overdose that requires immediate treatment. Second, these known devices give opportunity to the nerfarious to tamper with the urine sample. These individuals may attempt to tamper with their urine to receive a certain toxicology test result. For example, the urine from the individual may be swapped with “clean” urine prior to collection having the effect of cheating the toxicology test.

The present invention provides a collection container that removably receives a catheter. The collection container includes a drum having, one or more compartments for receiving test strips for completing an analyses of the collected urine quickly. Further, the present invention collects urine directly from the bladder of the individual and prevents certain types of tampering, such as utilizing “clean” urine as a replacement. In addition, the present invention provides a customizable toxicology screening that tests certain chemicals that may be completed at the bedside of the patient.

In light of the devices disclosed in the known art, it is submitted that the present invention substantially diverges in design elements from the known art and consequently it is clear that there is a need in the art for an improvement to urine toxicology testing devices. In this regard the instant invention substantially fulfills these needs.

### SUMMARY OF THE INVENTION

In view of the foregoing disadvantages inherent in the known types of urine testing devices now present in the known art, the present invention provides a urine testing device that utilizes a collection container that removably receives a catheter, herein a drum is configured to receive test strips therein. The present invention further provides a lid having a valve that is movable between an open and closed position.

It is therefore an object of the present invention to provide a new and improved urine testing device that has all of the advantages of the known art and none of the disadvantages.

It is another object of the present invention to provide a urine toxicology testing device that allows urine to be collected from patients directly from the bladder and without requiring the transferring of urine from one container to another.

It is another object of the present invention to provide a urine toxicology testing device having a drum that receives one or more testing strips within compartments positioned therein. The testing strips can test the urine within the container and detect drugs, their metabolites, and the like.

It is another object of the present invention to provide a urine toxicologyy testing device having a lid that is movable between an open position that allows urine to be transferred from the catheter into the container, and a closed position that allows the container and its contents to be sealed.

Other objects, features, anal advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner which it may be made and used may be better understood after a review of the following description, taken in connection with the accompanying drawings wherein like numeral annotations are provided throughout.

FIG. 1 shows a perspective view of one embodiment of the present invention.

FIG. 2 shows an exploded view of one embodiment of the present invention.

FIG. 3 shows an overhead view of one embodiment of the collection container of present invention.

FIG. 4 shows a phantom perspective view of one embodiment of the lid of the present invention.

FIG. 5A shows a cross sectional view of one embodiment of the present invention in an open position taken along 4-4 of FIG. 4 of the present invention.

FIG. 5B shows a cross sectional view of one embodiment of the present invention. in a closed position taken along 4-4 of FIG. 4 of the present invention.

### DETAILED DESCRIPTION OF THE INVENTION

Reference is made herein to the attached drawings. Like reference numerals are used throughout the drawings to depict like or similar elements of the urine a device. For the purposes of presenting a brief and clear description of the present invention, the preferred embodiment will be discussed as used for collecting urine from a patient. The figures are intended for representative purposes only and should not be considered to be limiting in any respect.

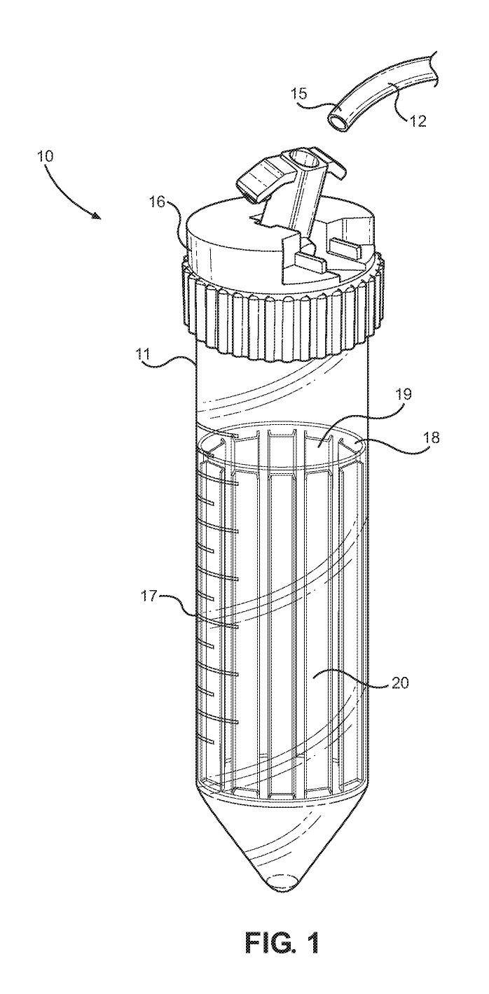

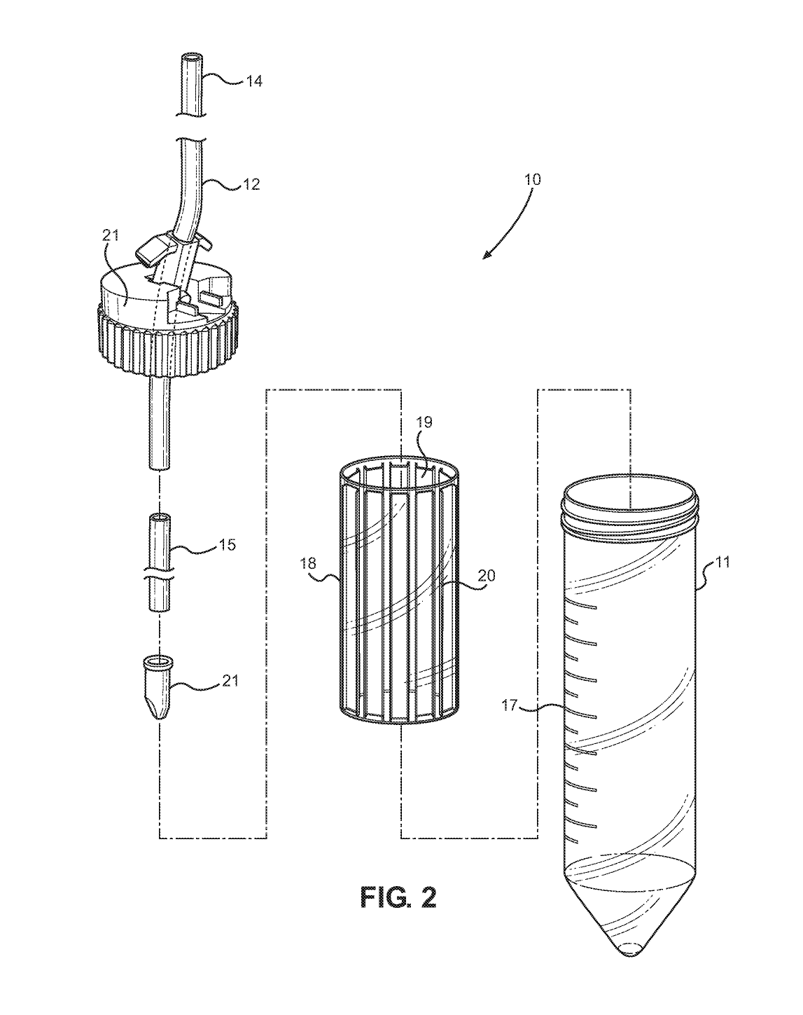

Referring now to FIGS. 1 and 2, there is shown a perspective view of one embodiment of the present invention and an exploded view of one embodiment of the present invention, respectfully. The present invention provides a urine testing device 10 that collects urine directly from an individual for the purpose of performing a chemical analysis of the collected urine. The urine testing device 10 comprises a collection container 11 that removably receives a catheter 12. The catheter 12 includes a first end 14 that is in fluid communication with an opposing second end 15, wherein the first end 13 is configured to be inserted into a urethra. The second end 15 is removably disposed within the collection container 11, through a lid 16 that is removably affixed to the collection container 11.

A drum 18 comprises one or more compartments 19 each configured to receive one or more test strips 20 therein for completing an analyses of the urine. The drum 18 is dimensioned to be received by and interior volume of the collection container 11, such that the urine collected in the container 11 contacts the one or more test strips 20.

In the shown embodiment, the collection container 11 is a centrifuge container having an open upper end that removably receives the lid 16 thereon. The drum 18 comprises a hollow cylinder having the one or more compartments 19 facing outward and disposed longitudinally therealong. The compartments 19 are substantially parallel relative to one another and spaced at intervals from adjacent compartments.

The collection container 11 further comprises spaced apart graduated measuring indicia 17 disposed on a sidewall of the collection container 11. Each indicia 17 is provided at the point of intersection of a plane coincidental with the horizontal level of fluid filling the collection container 11. In one embodiment, the indicia 17 is calibrated for indicating the volume of fluid within the container 11 with the drum 18. In another embodiment, the indicia 17 is calibrated for indicating the volume of fluid within the container 11 without the drum 18.

Referring specifically to FIG. 2, the catheter 12 includes a one-way valve 21 configured to prevent backflow of fluid from the second end 15 towards the first end 14. In this way, urine that is collected—Within the collection container 11 cannot reenter the body by flowing back up the catheter 12. Further, the valve 21 allows urine to flow through the catheter 12 without air pockets forming.

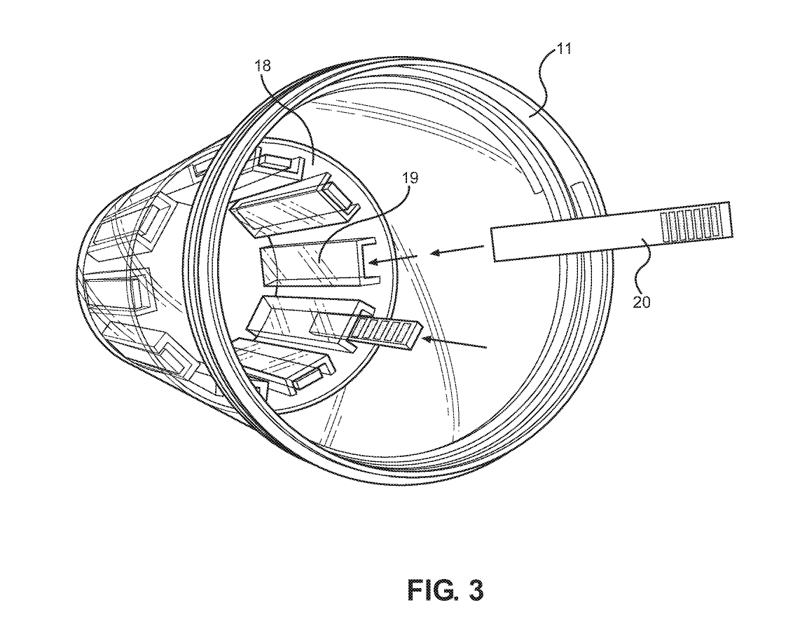

Referring now to FIG. 3, there is shown an overhead view of one embodiment of the collection container of present invention. In the shown embodiment, the drum 18 is received within the container 11 and testing strips 20 are being inserted into the one or more compartments 19. The container 11 and the drum 18 have corresponding dimensions, such that the drum 18 fits within the cylindrical container 11. However, in alternative embodiments, the container 11 and drum 18 may comprise any shape, such as rectangular, oval, and the like.

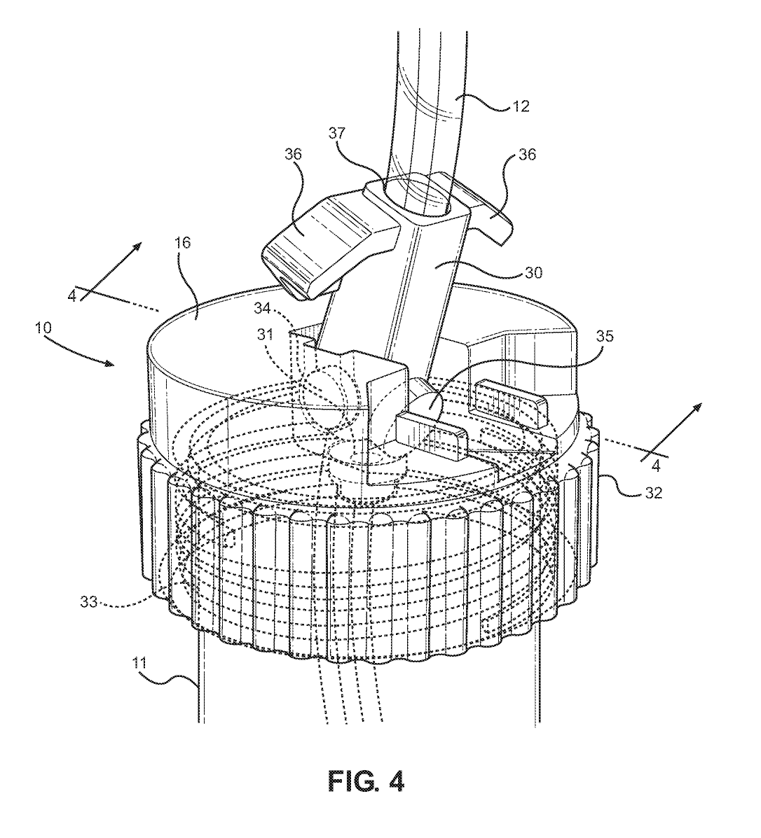

Referring now to FIG. 4, there is shown a phantom perspective view of one embodiment of the lid of the present invention. In the shown embodiment, the lid 16 includes ridges 32 disposed annularly about the perimeter thereof. The ridges 32 provide a gripping surface for removing and affixing the lid 16 to an upper end of the collection container 11. The lid 16 is threadably affixed to the upper end of the collection container 11 via corresponding threaded fasteners 33. The lid 16 includes a valve 30 that is hingedly affixed thereto and an aperture 31 that is configured to receive the catheter 12 therethrough. The valve 30 is hingedly affixed to the lid via hinge 34. In the shown embodiment, the hinge 34 is a ball and socket joint, wherein the lid 16 includes a pair of corresponding recessed sockets each configured to receive a ball member to permit rotation of the valve is such, the valve 30 is movable between an open position and a closed position.

The valve 30 further comprises a protruding member 35 dimensioned to cover the aperture 31 when in a closed position. A channel 37 runs through the valve 30 and sized to receive the catheter 12 therethrough. In the shown embodiment, the valve 30 includes one or more flanges 36 extending outwardly on opposing sides thereof.

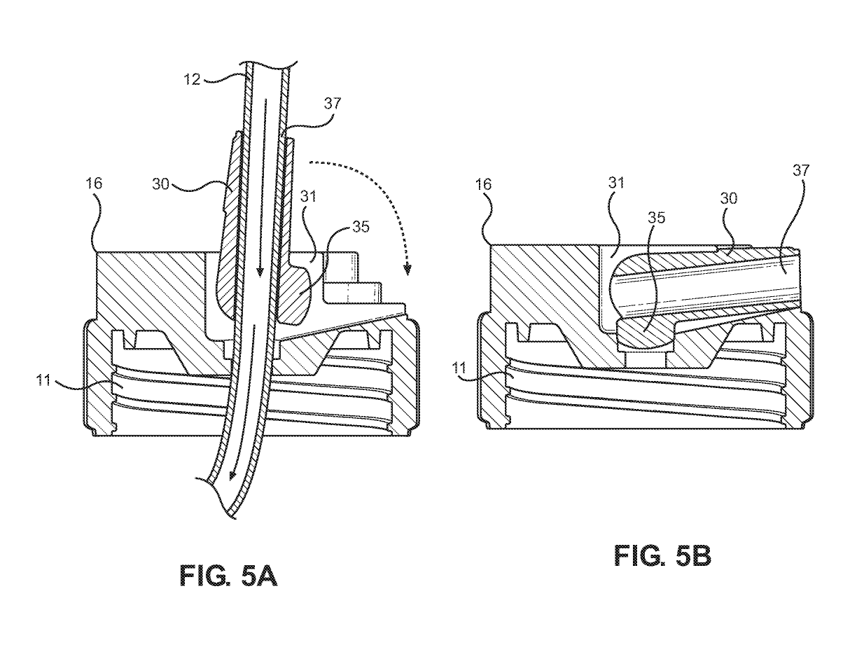

Referring now to FIGS. 5A, and 5B, there are shown cross sectional views of one embodiment of the present invention in an open position and a closed position taken along 4-4 of FIG. 4 of the present invention, respectfully. In the shown embodiments, the lid 16 is threadably affixed to the container 11. In FIG. 5A, the valve 30 is in an open position and the catheter 12 is running through the channel 37 and aperture 31. In the open position, the channel 37 is aligned over the aperture 31 to permit fluid to freely flow from the catheter 12 into the container 11.

In FIG. 5B, the valve is in a closed position and the catheter has been removed from the channel 37. The removal of the catheter 12 allows the valve 30 to rotate towards the container 11, wherein the upper surface of the valve 20 and lid 16 are substantially flush with each other. In the closed position, the protruding member 35 is positioned within the aperture 31 and prevents fluids from flowing therethrough.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A urine toxicology testing device comprising;
a collection container configured to store urine therein;
a catheter comprising a first end in fluid communication with an opposing second end;
wherein the first end of the catheter is configured to be inserted into a urethra and the second end is removably disposed within the collection container;
a lid removably affixed to the collection container, the lid including an aperture configured to receive the catheter therethrough;
a drum comprising one or more compartments configured to receive test strips; and
wherein the drum is dimensioned to be received by the collection container.

2. The urine toxicology testing device of claim 1, wherein:
the catheter includes a one-way valve configure to prevent backflow of fluid from the second end towards the first end.

3. The urine toxicology testing device of claim 1, wherein:
the drum comprises a hollow cylinder including one or more compartments facing outward and disposed longitudinally therealong;
wherein the one or more compartments are parallel relative to one another and spaced at intervals from adjacent compartments.

4. The urine toxicology testing device of claim 1, wherein:
the collection container includes measuring indicia for indicating the volume of fluid within the collection container.

5. The urine toxicology testing device of claim 1, wherein:
the lid includes ridges disposed annularly about a perimeter thereof;
wherein the ridges provide a gripping surface for removing and affixing the lid to an upper end of the collection container.

6. The urine toxicology testing device of claim 1, wherein:
the lid is threadably affixed to the collection container.

7. The urine toxicology testing device of claim 1, wherein:
a valve is hingedly affixed to the lid;
the valve comprising a protruding member dimensioned to cover the aperture of the lid and a channel configured to receive the catheter therethrough;
wherein the valve is movable between an open position and a closed position;
wherein the open position the channel s aligned with the aperture to perm e catheter to be positioned therethrough;
wherein the closed position the protruding member is aligned with the aperture to prevent fluid from passing therethrough.

8. The urine toxicology testing device of claim 8, wherein:
the valve includes one or more flanges extending outwardly on opposing sides thereof.

9. The urine toxicology testing device of claim 8, wherein:
the valve is hingedly affixed to the lid via a ball and socket joint;
wherein the valve comprises a pair of ball members disposed on opposing sides thereof;
wherein the lid comprises a pair of corresponding recesses; sockets configured to receive the ball members to permit rotation of the valve.
